(19) United States
(12) Patent Application Publication
(10) Pub. No.: US 2011/0263943 A1
(43) Pub. Date: Oct. 27, 2011

(54) ENDOSCOPE APPARATUS

(75) Inventor: Hiroshi YAMAGUCHI, Kanagawa (JP)

(73) Assignee: FUJIFILM CORPORATION, Tokyo (JP)

(21) Appl. No.: 13/093,567

(22) Filed: Apr. 25, 2011

(30) Foreign Application Priority Data
Apr. 26, 2010 (JP) 2010-101390

(51) Int. Cl. A61B 1/06

(52) U.S. Cl. 600/178

(57) ABSTRACT

An endoscope apparatus that captures images of a target using returned light from the target irradiated with irradiation light. The endoscope apparatus comprises a first optical system that has an axial chromatic aberration and respectively focuses light in different wavelength regions contained in the irradiation light at different positions on an optical axis thereof; a second optical system that focuses first returned light in a different wavelength region than light contained in the irradiation light and second returned light in a different wavelength region than the first returned light at substantially the same position on an optical axis thereof, the first returned light and the second returned light being returned from focal positions of the irradiation light focused in the target by the first optical system; and a light receiving section that receives the first returned light and the second returned light focused by the second optical system.

### BACKGROUND

1. Technical Field

The present invention relates to an endoscope apparatus. The contents of the following Japanese patent application are incorporated herein by reference, NO. 2010-101390 filed on Apr. 26, 2010.

2. Related Art

An observation apparatus is known that optically obtains information at different depths in an organism, as shown in Patent Documents 1 and 2, for example.
Patent Document 1: Japanese Patent Application Publication No. 2005-99430
Patent Document 2: Japanese Patent Application Publication No. 2007-47228

With fluorescent light observation, fluorescent light having a different wavelength than the irradiation light must be detected. When an objective lens is used to focus the irradiation light at different depths according to the wavelength and the wavelength of the observation light differs from the wavelength of the irradiation light, the position observed through the objective lens is different from the positions resulting from the different wavelengths of the irradiation light being focused at different depths.

### SUMMARY

In order to solve the above problems, according to a first aspect related to the innovations herein, provided is an endoscope apparatus that captures images of a target using returned light from the target irradiated with irradiation light. The endoscope apparatus comprises a first optical system that has an axial chromatic aberration and respectively focuses light in different wavelength regions contained in the irradiation light at different positions on an optical axis thereof; a second optical system that focuses first returned light in a different wavelength region than light contained in the irradiation light and second returned light in a different wavelength region than the first returned light at substantially the same position on an optical axis thereof, the first returned light and the second returned light being returned from focal positions of the irradiation light focused in the target by the first optical system; and a light receiving section that receives the first returned light and the second returned light focused by the second optical system.

The target may include a luminescent substance that emits luminescent light when excited by the light in at least one of the different wavelength regions in the irradiation light, and the second optical system may focus the first returned light, which is luminescent light emitted by the luminescent substance, and the second returned light at substantially the same position on the optical axis thereof.

The luminescent substance may be excited by the light in the different wavelength regions in the irradiation light to respectively emit first luminescent light and second luminescent light wavelength regions different from each other, and the second optical system may focus the first returned light and the second returned light, which are respectively the first luminescent light and the second luminescent light, at substantially the same position on the optical axis thereof.

The endoscope apparatus may further comprise a first wavelength filter that passes light in the wavelength region of the first returned light and a second wavelength filter that passes light in the wavelength region of the second returned light. The light receiving section may include a first light receiving element that receives light passed by the first wavelength filter and a second light receiving element that receives light passed by the second wavelength filter.

A plurality of the first wavelength filters and a plurality of the second wavelength filters may be arranged two-dimensionally, and a plurality of the first light receiving elements and a plurality of the second light receiving elements may be respectively arranged at positions corresponding to the first wavelength filters and the second wavelength filters.

The endoscope apparatus may further comprise an image generating section that generates images at the focal positions of the irradiation light in the target, based on the first returned light and the second returned light received by the light receiving section.

The endoscope apparatus may further comprise a light source that generates the irradiation light.

The light source may emit the irradiation light to include light in different wavelength regions for respectively exciting different luminescent substances to emit luminescent light in different wavelength regions.

The endoscope apparatus may further comprise an injecting section that injects the luminescent substance into the target.

The second optical system may be arranged such that the optical axis thereof has a different orientation than the optical axis of the first optical system.

The summary clause does not necessarily describe all necessary features of the embodiments of the present invention. The present invention may also be a sub-combination of the features described above.

### BRIEF DESCRIPTION OF THE DRAWINGS

FIG. 1 shows an exemplary endoscope apparatus 10 according to an embodiment of the present invention.

FIG. 2 is a schematic view of an exemplary configuration of the light transmission tube 280 together with the analyte 20.

FIG. 3 is a schematic view of an exemplary configuration of the image capturing section 124 together with the analyte 20.

FIG. 4 is a schematic view of exemplary configurations of the excitation light irradiating system and an image capturing system in the insertion section 120.

FIG. 5 is a schematic view of exemplary configuration of the light receiving section 320 and the wavelength filter section 330.

FIG. 6 shows exemplary image capturing timings of the illumination light images and the fluorescent light images by the image capturing section 124.

FIG. 7 shows an exemplary screen of the display apparatus 140.

FIG. 8 shows another exemplary fluorescent light image generated by the image generating section 102.

### DESCRIPTION OF EXEMPLARY EMBODIMENTS

Hereinafter, some embodiments of the present invention will be described. The embodiments do not limit the invention according to the claims, and all the combinations of the features described in the embodiments are not necessarily essential to means provided by aspects of the invention.

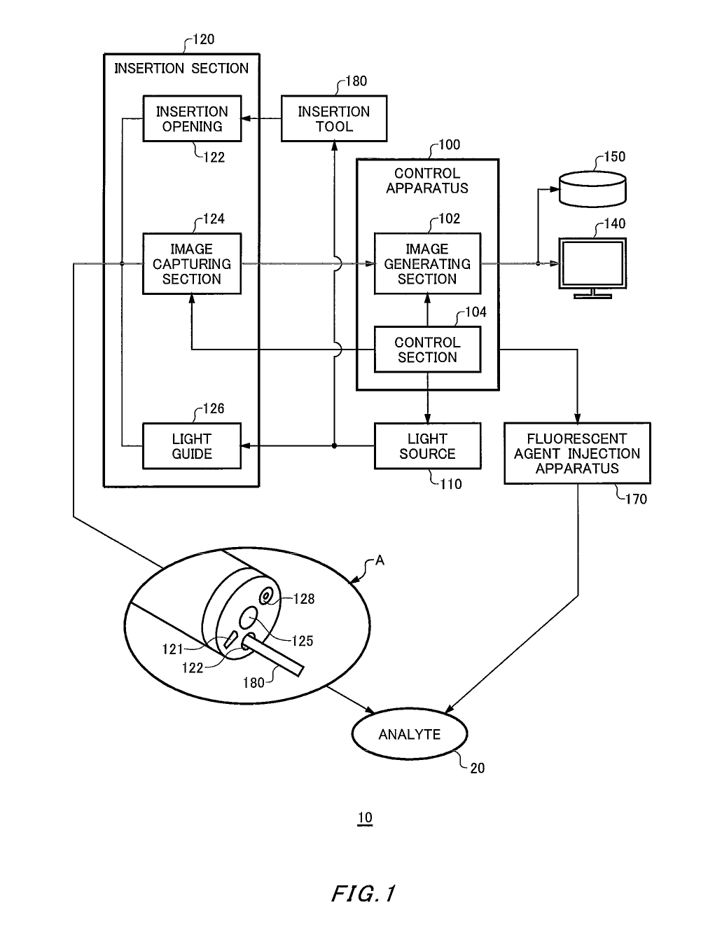

FIG. 1 shows an exemplary endoscope apparatus 10 according to an embodiment of the present invention. The endoscope apparatus 10 of the present embodiment captures an image of an analyte 20, which is a living creature, for example. Specifically, the endoscope apparatus 10 captures an image of the analyte 20 using returned light from the analyte 20 irradiated with irradiation light.

In the present embodiment, the endoscope apparatus 10 captures fluorescent light images within the analyte 20 at different depths. Specifically, the endoscope apparatus 10 may irradiate the analyte 20 with excitation light having different wavelengths through an irradiating optical system having an axial chromatic aberration. Inside the analyte 20, the excitation light is focused at different positions on the optical axis of the irradiating optical system depending on the wavelength. The analyte 20 contains a fluorescent material and the excitation light, in which each wavelength is focused differently, can excite the fluorescent substance at each position where a certain wavelength of the excitation light is focused. The fluorescent substance emits fluorescent light at each of these focal positions, and this fluorescent light becomes incident to the endoscope apparatus 10 as returned light. In other words, the excitation light emitted by the endoscope apparatus 10 is wavelength-converted and returned to the endoscope apparatus 10 as returned light.

The image capturing optical system of the endoscope apparatus 10 has an axial chromatic aberration such that the fluorescent light from each focal position in the analyte 20 is focused at substantially the same position on the optical axis of the image capturing optical system. The endoscope apparatus 10 can perform a one-shot capture of fluorescent light images at different depths by capturing a fluorescent light image of the analyte 20 via this image capturing optical system. The endoscope apparatus 10 of the present embodiment can perform one-shot image capturing of fluorescent light images at different depths, and provide the resulting image to an observer.

The analyte 20 in the present embodiment may be an internal organ such as an intestinal tube, including the stomach, large intestine, colon, or the like inside a living creature such as a person, for example. The analyte 20 may be the outside or the inside lining of an internal organ. In the present embodiment, the region serving as the image capturing target of the endoscope apparatus 10 is referred to as the analyte 20. The endoscope apparatus 10 includes an insertion section 120, a light source 110, a control apparatus 100, a fluorescent agent injection apparatus 170, a recording apparatus 150, a display apparatus 140, and an insertion tool 180. An enlarged view of the tip of the insertion section 120 is shown in section A of FIG. 1.

The insertion section 120 includes an insertion opening 122, an image capturing section 124, and a light guide 126. The tip of the insertion section 120 includes an objective lens 125 as a portion of the image capturing section 124. The objective lens 125 is included in the image capturing optical system. The tip of the insertion opening 122 includes a nozzle 121.

The insertion section 120 is inserted into an organism. A treatment tool, such as forceps, for treating the analyte 20 is inserted into the insertion opening 122. The treatment tool is an example of the insertion tool 180. The insertion opening 122 guides the insertion tool 180 inserted thereto to the tip. The insertion tool 180, which is exemplified by forceps, can have a variety of tip shapes. The nozzle 121 discharges water or air toward the analyte 20.

The light guide 126 guides the light emitted by the light source 110 to the irradiating section 128. The light guide 126 can be realized using optical fiber, for example. The irradiating section 128 emits the light guided by the light guide 126 toward the analyte 20. The image capturing section 124 receives the light returning from the analyte 20 via the objective lens 125 to capture an image of the analyte 20.

The image capturing section 124 can capture illumination light images of the analyte 20 using light emitted through the light guide 126. The image capturing section 124 captures an illumination light image of the analyte 20 using illumination light with a relatively broad spectrum in the visible light band. When capturing an illumination light image, the light source 110 emits substantially white light in the visible light region. The illumination light includes light in the red wavelength region, the green wavelength region, and the blue wavelength region, for example. The illumination light emitted by the light source 110 is emitted toward the analyte 20 from the irradiating section 128 via the light guide 126. The objective lens 125 receives, as the returned light, light in the visible light region expanded to have substantially the same wavelength region as the illumination light, as a result of the analyte 20 reflecting and scattering the illumination light. The image capturing section 124 captures an image via the objective lens 125 using the returned light from the analyte 20. The light source 110 may include an illumination light source that generates the illumination light. The illumination light source may be a discharge lamp such as a xenon lamp, a semiconductor light emitting element such as an LED, or the like.

In addition to the illumination light images, the image capturing section 124 can capture luminescent light images of the analyte 20. The luminescent light images are captured using luminescent light, which is an example of returned light from the analyte 20. Fluorescent and phosphorescent light are included in the scope of the luminescent light. In the present embodiment, the image capturing section 124 captures images via the objective lens 125 using luminescent light generated by photoluminescence as a result of excitation light or the like, which is an example of irradiation light. In particular, the image capturing section 124 captures fluorescent light images as examples of luminescent images, using fluorescent light generated via photoluminescence.

When capturing a fluorescent light image of the analyte 20, the light source 110 generates excitation light. The excitation light generated by the light source 110 is emitted toward the analyte 20 from the tip of the insertion opening 122, via an excitation light guide provided in the light transmission tube, as an example of the insertion tool 180. The analyte 20 includes a fluorescent substance, as an example of a luminescent substance, and this fluorescent substance is excited by the excitation light to emit fluorescent light in a different wavelength region than the excitation light. For example, the analyte 20 may emit fluorescent light in a longer wavelength region than the excitation light. The image capturing section 124 captures a fluorescent light image of the analyte 20 using the fluorescent returned light. The light source 110 may include an excitation light source that generates the excitation light. The excitation light source may be a semiconductor light emitting element such as an LED or a diode laser. As other examples, the excitation light source can use a laser with a variety of lasing media such as a diode laser, a fixed laser, or a liquid laser.

The excitation light passes through an irradiating optical system, which is different from the image capturing optical system, to irradiate the analyte 20. The irradiating optical system, which is a portion of the excitation light guide, is provided on the tip of the light transmission tube. The excitation light passes through the irradiating optical system to irradiate the analyte 20. The excitation light in the present embodiment includes a plurality of components in different wavelength regions. The irradiating optical system has an axial chromatic aberration. Due to the axial chromatic aberration of the irradiating optical system, the light components in the different wavelength regions included in the excitation light are focused at different positions on the optical axis. In order to focus each light component of the excitation light together in the analyte 20, the analyte 20 is irradiated by the excitation light with the position of the light transmission tube being fixed in the insertion opening 122.

The fluorescent substance contained in the analyte 20 can be excited by any of the light components of the excitation light. The fluorescent substance may be injected into the analyte 20 from the outside. For example, the fluorescent substance may be injected into the analyte 20 by the fluorescent agent injection apparatus 170. In the present embodiment, the image capturing section 124 captures images of the analyte 20 using fluorescent light from a plurality of different types of fluorescent substances contained in the analyte 20.

The first fluorescent substance may be indo cyanine green (ICG), for example. The fluorescent agent injection apparatus 170 may inject the ICG into the blood vessels of an organism using an intravenous injection. The amount of ICG that the fluorescent agent injection apparatus 170 injects into the analyte 20 is controlled by the control apparatus 100 to maintain a substantially constant concentration of ICG in the organism. The ICG is excited by infrared rays with a wavelength of 780 nm, for example, and generates fluorescent light whose primary spectrum is in a wavelength region of 830 nm. In the present embodiment, the image capturing section 124 captures fluorescent light images of the analyte 20 using the fluorescent light generated by the ICG, which is the first luminescent substance.

The second fluorescent substance can be a fluorescent substance contained in structural components, such as cells, of the analyte 20. This fluorescent substance contained in the analyte 20 may be reduced NADH (nicotinamide adenine dinucleotide), for example. NADH is excited by light with a wavelength of 340 nm in the ultra violet wavelength region to emit fluorescent light whose primary spectrum is in the 450 nm wavelength region. In this way, the image capturing section 124 can capture fluorescent light images of the analyte 20 using the organism's own fluorescent light.

When the image capturing section 124 captures the fluorescent light images, the light source 110 emits excitation light having a 340 nm wavelength region as the primary component and excitation light having a 780 nm wavelength region as the primary component. The excitation light emitted by the light source 110 passes through the irradiating optical system having the axial chromatic aberration such that each light component is focused at a different position in the analyte 20. As a result, fluorescent light mostly in a 450 nm wavelength region and fluorescent light mostly in an 830 nm wavelength region are emitted from different positions within the analyte 20. The image capturing optical system, which has a different axial chromatic aberration than the irradiating optical system, focuses these two types of fluorescent light at substantially the same position on the optical axis of the image capturing optical system. The image capturing section 124 can capture the fluorescent light images at different positions in the analyte 20 by receiving the fluorescent light with light receiving elements provided at this focal position.

In addition to NADH, the fluorescent substance in an organism that emits fluorescent light for the image capturing may be FAD (flavin adenine dinucleotide), for example. Each type of fluorescent substance may be injected into the analyte 20 from the outside or may be already present in the analyte 20. The fluorescent substance may be a combination of a fluorescent substance injected into the analyte 20 from the outside and a fluorescent substance already present in the analyte 20. Three or more types of fluorescent substances may be used. If a fluorescent substance is used that emits fluorescent light in different wavelength regions as a result of being excited by excitation light in different wavelength regions, the image capturing section 124 can capture the fluorescent light images using only the fluorescent light from the fluorescent substance.

When the analyte 20 is irradiated and light in a wavelength region different from the wavelength region of the irradiation light from the focal positions in the analyte 20 is returned to the endoscope apparatus 10, the image capturing section 124 can capture images of the analyte 20 using this returned light. The process for generating luminescent light may be photoluminescence, chemical luminescence, or thermoluminescence, for example. If the luminescent light is generated from the analyte 20 indirectly using a chemical process and/or a thermal process when the analyte 20 is irradiated, the image capturing section 124 can capture an image of the analyte 20 using this luminescent light.

The control apparatus 100 includes an image generating section 102 and a control section 104. The control section 104 controls the image capturing section 124 and the light source 110 and uses the image capturing section 124 to capture the illumination light images and the fluorescent light images. Specifically, the control section 104 causes the image capturing section 124 to switch over time between capturing the illumination light images and capturing the fluorescent light images.

The image generating section 102 generates an output image to be output to the outside, based on the illumination light images and the fluorescent light images captured by the image capturing section 124. For example, the image generating section 102 may output the generated output image to at least one of the recording apparatus 150 and the display apparatus 140. More specifically, the image generating section 102 generates an image from the plurality of images captured by the image capturing section 124, and outputs this image to at least one of the recording apparatus 150 and the display apparatus 140. The image generating section 102 may output the output image to at least one of the recording apparatus 150 and the display apparatus 140 via a communication network such as the Internet.

The display apparatus 140 displays images including the fluorescent light images and the illumination light images generated by the image generating section 102. The recording apparatus 150 records the fluorescent light images and the illumination light images generated by the image generating section 102 in a non-volatile recording medium. For example, the recording apparatus 150 may store the images in a magnetic recording medium such as a hard disk or in an optical recording medium such as an optical disk.

The endoscope apparatus 10 described above can receive fluorescent light from different depths in the analyte 20 with a single exposure. Therefore, fluorescent light images at different depths in the analyte 20 can be captured with a single shot. Furthermore, the endoscope apparatus 10 can provide an observer with a fluorescent light image in which positional relationships in the depth direction are easily understood.

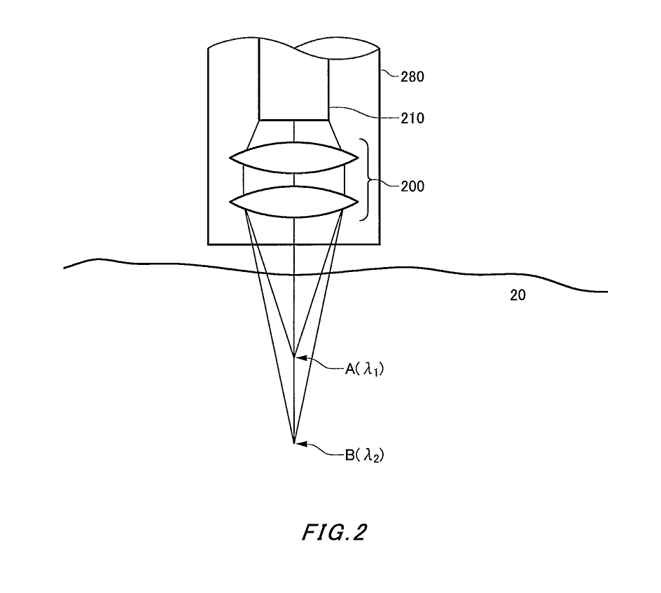

FIG. 2 is a schematic view of an exemplary configuration of the light transmission tube 280 together with the analyte 20. The light transmission tube 280 contains the irradiating optical system 200 and a light guide tube 210 serving as an excitation light guide. The light guide tube 210 guides excitation light that is a combination of light in a wavelength region of 340 nm (λ₁) emitted by the light source 110 and light in a wavelength region of 780 nm (λ₂) emitted by the light source 110. The light guide tube 210 can be realized by optical fiber.

The excitation light guided by the light guide tube 210 passes through the irradiating optical system 200 having an axial chromatic aberration and irradiates the analyte 20. In the irradiating optical system 200, the spherical aberration for the wavelength region of the excitation light is significantly less than the axial chromatic aberration. The irradiating optical system 200 substantially focuses the λ₁ light component in the excitation light at point A on the optical axis of the irradiating optical system 200. The irradiating optical system 200 substantially focuses the λ₂ light component in the excitation light at point B, which is different from point A, on the optical axis of the irradiating optical system 200. In order to position both point A and point B within the analyte 20, the excitation light is emitted while the light transmission tube 280 is aligned with these points within the insertion opening 122.

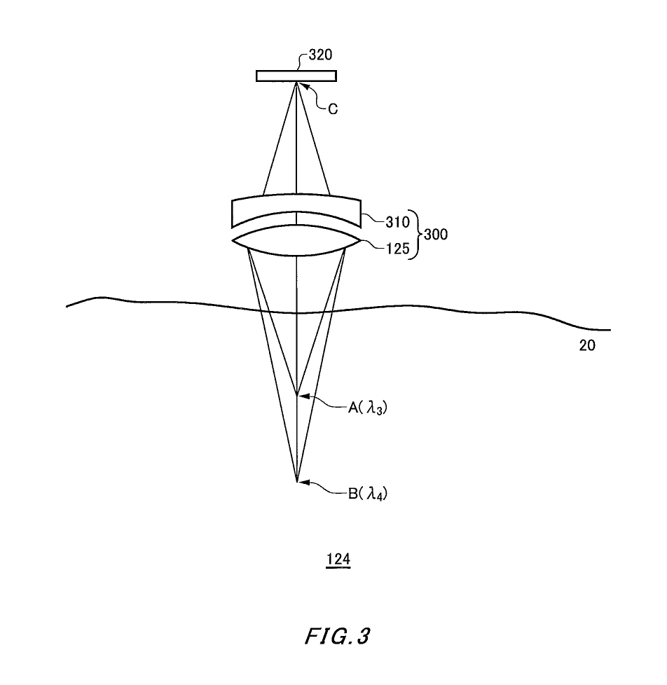

FIG. 3 is a schematic view of an exemplary configuration of the image capturing section 124 together with the analyte 20. The image capturing section 124 includes an image capturing optical system 300 and a light receiving section 320. The image capturing optical system 300 includes the objective lens 125 and a chromatic aberration correcting optical system 310. Here, in order to clearly describe the endoscope apparatus 10, point A and point B are positioned on the optical axis of the image capturing optical system 300. The following description focuses on the light receiving section 320 and the image capturing optical system 300 of the image capturing section 124.

The λ₁ light component focused at point A by the irradiating optical system 200 excites NADH present at point A. The excited NADH emits fluorescent light in a 450 nm (λ₃) wavelength region. The λ₂ light component focused at point B by the irradiating optical system 200 excites ICG present at point B. The excited ICG emits fluorescent light in a 830 nm (λ₄) wavelength region. The λ₃ fluorescent light is an example of first returned light, and the λ₄ fluorescent light is an example of second returned light.

The image capturing optical system 300 has optical characteristics to substantially focus both the λ₃ light emitted from point A and the λ₄ light emitted from point B at point C. Specifically, the chromatic aberration of the chromatic aberration correcting optical system 310 is adjusted with respect to the λ₃ light from point A and the λ₄ light from point B. Here, Z represents the positional difference between the position on the optical axis of the image capturing optical system 300 at which the λ₃ light from point A is focused by the image capturing optical system 300 and the position on the optical axis of the image capturing optical system 300 at which the λ₄ light from point B is focused by the image capturing optical system 300. The chromatic aberration correcting optical system 310 may be any optical system that can decrease the Z value of the image capturing optical system 300 more than if the chromatic aberration correcting optical system 310 were not used.

The light receiving section 320 is provided near point C in the direction of the optical axis of the image capturing optical system 300. As a result, the light receiving elements of the light receiving section 320 near point C can receive the λ₃ fluorescent light and the λ₄ fluorescent light focused by the image capturing optical system 300. In other words, the light receiving section 320 can receive the λ₃ fluorescent light and the λ₄ fluorescent light focused by the image capturing optical system 300.

If the analyte 20 is a medium with light scattering properties, such as a living organism, the light components focused toward point A and point B are scattered by the analyte 20. Therefore, each light component widens a certain amount around the focal point. As a result, the light receiving section 320 can receive fluorescent light from regions resulting from the widening of each light component. Therefore, the image capturing section 124 can capture a fluorescent light image of the region near point A and a fluorescent light image of the region near point B, with a single image capture.

As described above, the irradiating optical system 200 focuses light in different wavelength regions contained in the excitation light at different positions on the optical axis. The fluorescent substances in the analyte 20 are respectively excited by the light in the different wavelength regions within the excitation light to emit fluorescent light in different wavelength regions. The image capturing optical system 300 focuses both the first returned light and the second returned light, which are fluorescent light, at substantially the same position on the optical axis of the image capturing optical system 300.

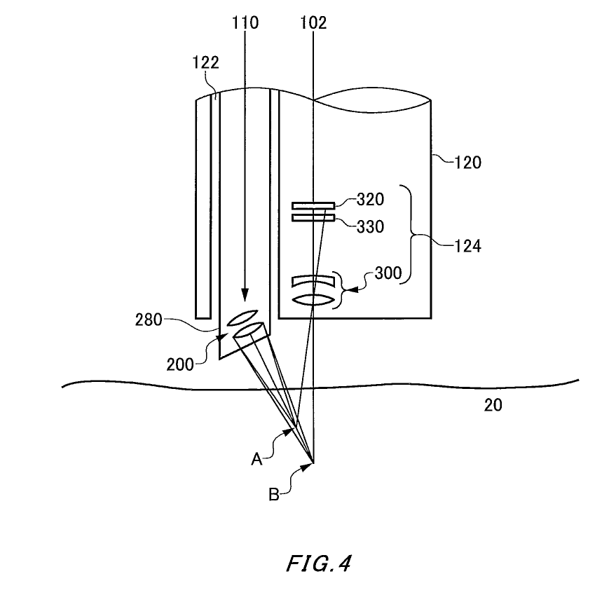

FIG. 4 is a schematic view of exemplary configurations of the excitation light irradiating system and an image capturing system in the insertion section 120. The position of the light transmission tube 280 is fixed near the surface of the analyte 20 by the insertion opening 122. The position of the tip of the light transmission tube 280 may be fixed as a result of contacting the surface of the analyte 20. The excitation light from the light source 110 passes through the irradiating optical system 200 to irradiate the analyte 20. The λ₄ light component in the excitation light is focused toward point A, and the λ₂ light component in the excitation light is focused toward point B.

The image capturing section 124 includes the light receiving section 320 and a wavelength filter section 330. The image capturing optical system 300 has optical characteristics to focus the λ₃ light from point A and the λ₄ light from point B at substantially the same position on the optical axis thereof. The light receiving section 320 is positioned at the focal position of the image capturing optical system 300. The wavelength filter section 330 is provided near the light receiving section 320 in the optical path of the returned light between the image capturing optical system 300 and the light receiving section 320. The wavelength filter section 330 has light transmission characteristics to pass at least the λ₃ light and the λ₄ light. The wavelength filter section 330 preferably has light transmission characteristics to substantially block the λ₁ light and the λ₂ light in the excitation light.

As shown in FIG. 4, the image capturing optical system 300 is arranged to have a different optical axis from the irradiating optical system 200. However, it should be noted that the optical axis of the irradiating optical system 200 is not orthogonal to the optical axis of the image capturing optical system 300, and the focal position of the image capturing optical system 300 for each light component is a different position on the optical axis of the irradiating optical system 200.

The λ₃ fluorescent light emitted from point A passes through the image capturing optical system 300 to be focused at the light receiving section 320. The λ₄ fluorescent light emitted from point B also passes through the image capturing optical system 300 to be focused at the light receiving section 320. The λ₃ fluorescent light generated near point A is received by the light receiving section 320 as a λ₃ fluorescent light image. The λ₄ fluorescent light generated near point B is received by the light receiving section 320 as a λ₄ fluorescent light image. The received light signals, which indicate the light received by the light receiving elements of the light receiving section 320, are supplied to the image generating section 102 as image capture signals.

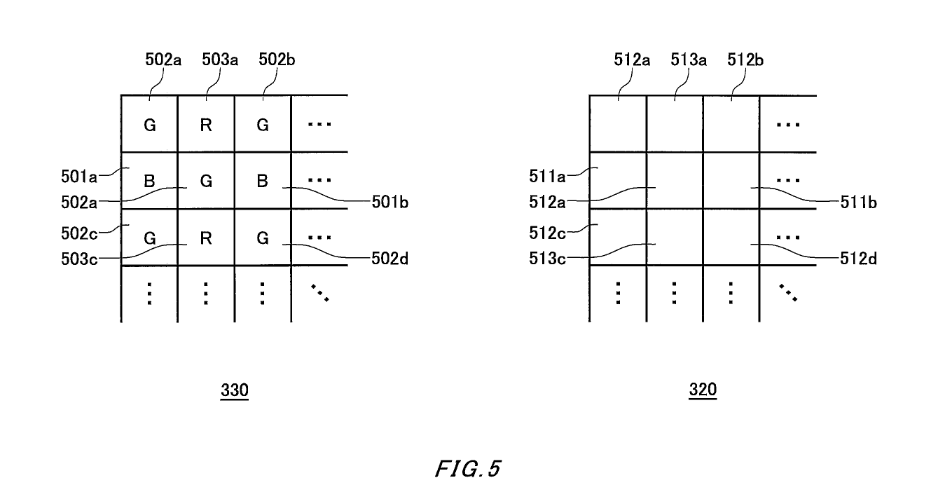

FIG. 5 is a schematic view of exemplary configurations of the light receiving section 320 and the wavelength filter section 330. The wavelength filter section 330 includes a plurality of blue light passing filters 501 that selectively pass light in the blue wavelength region, a plurality of green light passing filters 502 that selectively pass light in the green wavelength region, and a plurality of red light passing filters 503 that pass at least light in the red wavelength region.

In FIG. 5, the blue light passing filters 501a and 501b, green light passing filters 502a to 502d, and red light passing filters 503a and 503c are shown. The blue light passing filter 501a, the two green light passing filters 502a, and the red light passing filter 503a are arranged in a matrix to form one wavelength filter unit. The wavelength filter section 330 may have a wavelength filter array in which a plurality of such wavelength filter units are arranged in a matrix, in the same manner as the light passing filters within a wavelength filter unit. In this way, the wavelength filter section 330 can be formed by arranging blue light passing filters 501, green light passing filters 502, and red light passing filters 503 in a two-dimensional array.

The light receiving section 320 may be formed by arranging a plurality of light receiving elements at positions to selectively receive light passed by the blue light passing filters 501, the green light passing filters 502, and the red light passing filters 503. Specifically, the light receiving section 320 may have a light receiving element array in which a plurality of blue light receiving sections 511 that selectively receive light in the blue wavelength region, a plurality of green light receiving sections 512 that selectively receive light in the green wavelength region, and a plurality of red light receiving sections 513 that receive at least light in the red wavelength region are arranged two-dimensionally.

More specifically, a blue light receiving section 511a receives light passed by a blue light passing filter 501a, a green light receiving section 512a receives light passed by a green light passing filter 502a, and a red light receiving section 513a receives light passed by a red light passing filter 503a. In this way, the blue light receiving sections 511, green light receiving sections 512, and red light receiving sections 513 can respectively be positioned to correspond to blue light passing filters 501, green light passing filters 502, and red light passing filters 503. Each light receiving element may be an image capturing element, such as a CCD or a CMOS.

Here, in addition to light in the red wavelength region, the red light passing filters 503 can also pass the wavelength region of the fluorescent light emitted by the ICG. In other words, the red light passing filters 503 selectively pass light in the red wavelength region and in the fluorescent light wavelength region emitted by the ICG. Therefore, when the analyte 20 is irradiated with excitation light, the fluorescent light emitted by the ICG can be received by the red light receiving sections 513 through the red light passing filters 503. Accordingly, the image capturing section 124 can use the red light receiving sections 513 to capture the fluorescent light images with the λ₄ fluorescent light. Furthermore, the fluorescent light emitted by NADH can be received by the blue light receiving sections 511 through the blue light passing filters 501. Accordingly, the image capturing section 124 can use the blue light receiving sections 511 to capture the fluorescent light images with the λ₃ fluorescent light.

If the irradiating section 128 emits illumination light spanning substantially the entire wavelength region of visible light, the image capturing section 124 can generate illumination light images of visible light using the blue light receiving sections 511, the green light receiving sections 512, and the red light receiving sections 513.

As described above, when the fluorescent substances are NADH and ICG, the blue light passing filters 501 can function as first wavelength filters that pass light in the wavelength region of the first returned light and the red light passing filters 503 can function as second wavelength filters that pass light in the wavelength region of the second returned light. Furthermore, the blue light receiving sections 511 can function as first light receiving elements that receive the light passed by the first wavelength filters and the red light receiving sections 513 can function as second light receiving elements that receive the light passed by the second wavelength filters.

The image generating section 102 generates images at the excitation light focal positions in the analyte 20, based on the λ₃ fluorescent light and the λ₄ fluorescent light received by the light receiving section 320. Specifically, the image generating section 102 generates fluorescent light images using the λ₃ fluorescent light based on image capture signals of the blue light receiving sections 511 that received the λ₃ fluorescent light. The fluorescent light images captured using the λ₃ fluorescent light are images showing the focal position of the λ₁ light component. The image generating section 102 generates fluorescent light images using the λ₄ fluorescent light based on image capture signals of the red light receiving sections 513 that received the λ₄ fluorescent light. The fluorescent light images captured using the λ₄ fluorescent light are images showing the focal position of the λ₂ light component.

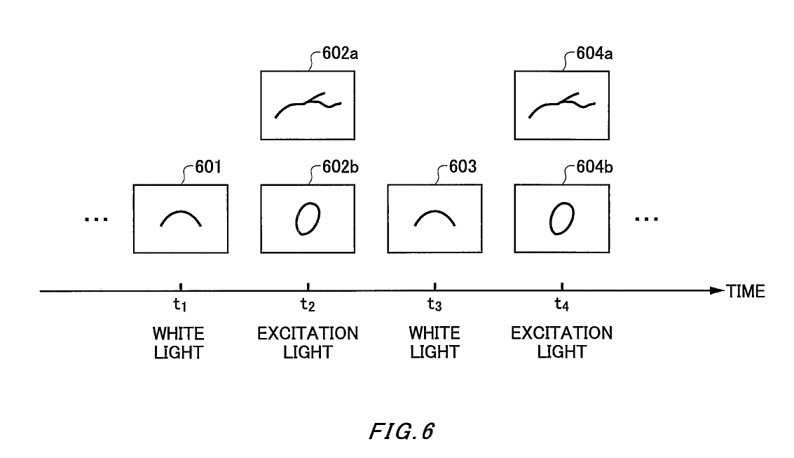

FIG. 6 shows exemplary image capturing timings of the illumination light images and the fluorescent light images by the image capturing section 124. The image capturing section 124 is controlled by the control section 104 to switch over time between capturing illumination light images and capturing fluorescent light images. In the example of FIG. 6, the image capturing section 124 captures an illumination light image 601, fluorescent light images 602, an illumination light image 603, fluorescent light images 604, etc. at the image capturing times t1, t2, t3, t4, etc.

During the exposure period at the image capturing timing of t1, the control section 104 causes white light to be irradiated as illumination light from the irradiating section 128 toward the analyte 20. When this exposure period is finished, the control section 104 switches the irradiation light from the white illumination light to the excitation light, and causes the excitation light to be irradiated toward the analyte 20 through the irradiating optical system 200 during the exposure period at the image capturing timing of t2.

Next, the control section 104 switches the irradiation light from the excitation light to white illumination light, and causes the white illumination light to be irradiated from the irradiating section 128 toward the analyte 20 during the exposure period at the image capturing timing of t3. After this, the control section 104 switches the irradiation light from the white illumination light to the excitation light, and causes the excitation light to be irradiated through the irradiating optical system 200 toward the analyte 20 during the exposure period at the image capturing timing of t4. As a result of the control section 104 repeating the irradiation light switching operation, the analyte 20 is alternately irradiated by illumination light and excitation light over time.

The control section 104 exposes the light receiving section 320 to the image capturing section 124 at each exposure period from t1 to t4, and outputs the acquired image capture signals from the light receiving section 320 to the image generating section 102. The image generating section 102 generates the illumination light image 601 based on the image capture signals from each of the blue light receiving sections 511, green light receiving sections 512, and red light receiving sections 513 acquired at the image capturing timing t1. The image generating section 102 generates the fluorescent light image 602b using the λ₃ fluorescent light, based on the image capture signals of the blue light receiving sections 511 acquired at the image capturing timing t2, and generates the fluorescent light image 602a using the λ₄ fluorescent light, based on the image capture signals of the red light receiving sections 513 acquired at the image capturing timing t2.

Next, the image generating section 102 generates the illumination light image 603 based on the image capture signals from each of the blue light receiving sections 511, green light receiving sections 512, and red light receiving sections 513 acquired at the image capturing timing t3. The image generating section 102 generates the fluorescent light image 604b using the λ₃ fluorescent light, based on the image capture signals of the blue light receiving sections 511 acquired at the image capturing timing t4, and generates the fluorescent light image 604a using the λ₄ fluorescent light, based on the image capture signals of the red light receiving sections 513 acquired at the image capturing timing t4.

When switching the irradiation light from the illumination light to the excitation light, the control section 104 may continue to drive the visible light source to emit light and insert, into the optical path from the visible light source, an illumination light cutoff filter that blocks the illumination light, thereby preventing irradiation with the illumination light from the irradiating section 128. The illumination light cutoff filter may be a filter that blocks at least light in the visible light region, and may be a light blocking filter that does not substantially pass light. Similarly, switching the irradiation light to the excitation light can be achieved by controlling an excitation light cutoff filter. The illumination light cutoff filter and the excitation light cutoff filter can be realized by filters whose light transmission characteristics can be electrically controlled, such as liquid crystal filters. The control section 104 can switch the irradiation light by electrically controlling the light transmission characteristics of the filters. When switching the irradiation light from the illumination light to the excitation light, the control section 104 may stop driving the LED serving as the illumination light source and drive the LED serving as the excitation light source. When switching the irradiation light from the excitation light to the illumination light, the control section 104 may stop driving the LED serving as the excitation light source and drive the LED serving as the illumination light source.

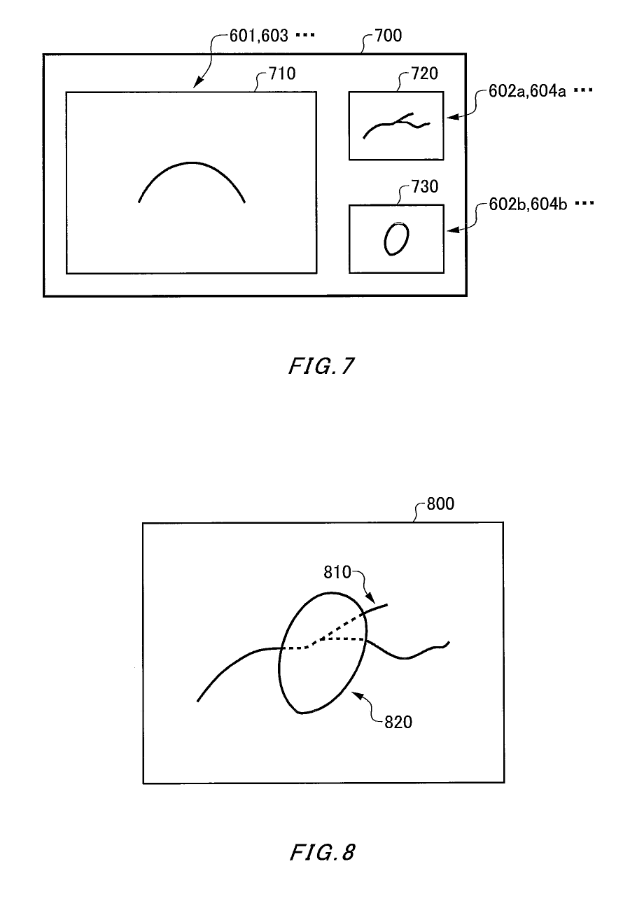

FIG. 7 shows an exemplary screen of the display apparatus 140. The image generating section 102 generates a view in the display area 710 of the screen 700 of the display apparatus 140 that sequentially changes between the illumination light image 601, the illumination light image 603, etc. Furthermore, the image generating section 102 generates a view in the display area 720 of the screen 700 of the display apparatus 140 that sequentially switches between the fluorescent light image 602a, the fluorescent light image 604a, etc. and a view in the display area 730 of the screen 700 of the display apparatus 140 that sequentially switches between the fluorescent light image 602b, the fluorescent light image 604b, etc.

The observer can observe a natural image such as seen by the naked eye from the tip of the insertion section 120, using the visible light view displayed in the display area 710. The observer can be made aware of blood vessels in the analyte 20 by the fluorescent light image 602a displayed in the display area 720. The observer can be made aware of a fluorescent light intensity distribution from NADH at a position that is shallower than the blood vessels mentioned above, by the fluorescent light image 602b displayed in the display area 730. By viewing the fluorescent light image 602b, the observer can recognize portions with relatively low fluorescent light intensity as being tumor tissue, for example.

The image generating section 102 may generate the fluorescent light image 602b and the fluorescent light image 602a in different colors. For example, the image generating section 102 may generate the fluorescent light image 602b such that the intensity of the received λ₃ fluorescent light is indicated by the strength of a first color and generate the fluorescent light image 602a such that the intensity of the received λ₄ fluorescent light is indicated by the strength of a second color. The first color may be a bluish color and the second color may be a reddish color, for example. When viewing the organism with the naked eye, objects on the top layer may appear blue. Therefore, by using a bluish color to represent the fluorescent light image 602b obtained when focusing on objects closer to the surface and using a reddish color to represent the fluorescent light image 602a obtained when focusing on deeper objects, the observer can see a fluorescent light image that seems natural.

FIG. 8 shows another exemplary fluorescent light image generated by the image generating section 102. The image generating section 102 may generate the composite image 800 based on the fluorescent light image 602a and the fluorescent light image 602b, by superimposing the fluorescent light image 602a and the fluorescent light image 602b on each other. The image generating section 102 may supply the composite image 800 to at least one of the display apparatus 140 and the recording apparatus 150 as an image to be displayed.

The image generating section 102 may generate the composite image 800 such that an object 820 extracted based on the image content of the fluorescent light image 602b is emphasized more than an object 810 extracted based on the image content of the fluorescent light image 602a. For example, the image generating section 102 may generate the composite image 800 such that the pixel values representing the object 810 are given more weight than the pixel values representing the object 820. Specifically, with I1(x, y) representing the pixel value of each pixel in the fluorescent light image 602b and I2(x, y) representing the pixel value of each pixel in the fluorescent light image 602a, the image generating section 102 may calculate corresponding pixel values I(x, y) in the composite image 800 such that I(x, y)=α×I1(x, y)+β×I2(x, y), where α>β.

More specifically, the image generating section 102 may overwrite the object 810 with the object 820. As a result, the composite image 800 can be generated to appropriately show the overlapping state of the objects in the analyte 20. When generating the composite image 800, the above process for emphasizing the object 820 more than the object 810 is particularly useful at borders between the object 810 and the object 820. With this process, the vertical positional relationship of the object 810 and the object 820 can be appropriately displayed in the composite image 800, and the observer can be clearly shown that the blood vessel or the like represented by the object 810 is deeper than the object 820.

The image generating section 102 may generate the composite image 800 such that the fluorescent light image 602b and the fluorescent light image 602a have different colors. For example, the image generating section 102 may generate the composite image 800 such that fluorescent light image 602b is represented by pixel values of a first color and the fluorescent light image 602a is represented by pixel values of a second color. Here as well, the first color may be a bluish color and the second color may be a reddish color. For a composite image 800 using different colors, the image generating section 102 may emphasize the object 820 more than the object 810. For example, the image generating section 102 may generate the composite image 800 such that the pixel values representing the object 820 are given more weight than the pixel values representing the object 810. The image generating section 102 may generate the composite image 800 such that the object 810 is overwritten by the object 820.

In the above description, the excitation light emitted by the light source 110 includes light in different wavelength regions, and the light in different wavelength regions respectively excites different fluorescent substances so that the fluorescent substances respectively emit fluorescent light in different wavelength regions. As another example, the first returned light component in the returned light may be fluorescent light and the other returned light component may be a type of returned light other than fluorescent light. Specifically, the second returned light may be light resulting from the reflection and/or scattering of the irradiation light.

In other words, the fluorescent substances included in the analyte 20 may be excited by at least the light in one of the different wavelength regions in the irradiation light to emit the fluorescent light. The image capturing optical system 300 may then focus the first returned light, which is fluorescent light, and the second returned light at substantially the same position on the optical axis.

If the wavelength of at least one light component having a different wavelength in the irradiation light is converted and used as the returned light, the returned light is not limited to fluorescent light. In other words, if first returned light in a different wavelength region than the light included in the irradiation light is returned from a first focal position of the irradiation light focused in the analyte 20 by the irradiating optical system 200 and second returned light in a different wavelength region than the first returned light is returned from a second focal position of the irradiation light, the image capturing section 124 can capture images using the first returned light and the second returned light. In this case, the image capturing optical system 300 focuses the first returned light in a different wavelength region than the light included in the irradiation light and the second returned light in a different wavelength region than the first returned light, which are returned from focal positions of the irradiation light focused in the analyte 20 by the irradiating optical system 200, at substantially the same position on the optical axis of the image capturing optical system 300.

The function of the control apparatus 100 described above may be realized by a computer. Specifically, by installing a program realizing the function of the control apparatus 100 in a computer, the computer may function as the image generating section 102 and the control section 104. This program may be stored in a computer readable storage medium such as a CD-ROM or a hard disk, and may be provided to the computer by having the computer read this storage medium. Instead, the program may be provided to the computer via a network.

While the embodiments of the present invention have been described, the technical scope of the invention is not limited to the above described embodiments. It is apparent to persons skilled in the art that various alterations and improvements can be added to the above-described embodiments. It is also apparent from the scope of the claims that the embodiments added with such alterations or improvements can be included in the technical scope of the invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

What is claimed is:

1. An endoscope apparatus that captures images of a target using returned light from the target irradiated with irradiation light, the endoscope apparatus comprising:
a first optical system that has an axial chromatic aberration and respectively focuses light in different wavelength regions contained in the irradiation light at different positions on an optical axis thereof;
a second optical system that focuses first returned light in a different wavelength region than light contained in the irradiation light and second returned light in a different wavelength region than the first returned light at substantially the same position on an optical axis thereof, the first returned light and the second returned light being returned from focal positions of the irradiation light focused in the target by the first optical system; and
a light receiving section that receives the first returned light and the second returned light focused by the second optical system.

2. The endoscope apparatus according to claim 1, wherein
the target includes a luminescent substance that emits luminescent light when excited by the light in at least one of the different wavelength regions in the irradiation light, and
the second optical system focuses the first returned light, which is luminescent light emitted by the luminescent substance, and the second returned light at substantially the same position on the optical axis thereof.

3. The endoscope apparatus according to claim 2, wherein
the luminescent substance is excited by the light in the different wavelength regions in the irradiation light to respectively emit first luminescent light and second luminescent light wavelength regions different from each other, and
the second optical system focuses the first returned light and the second returned light, which are respectively the first luminescent light and the second luminescent light, at substantially the same position on the optical axis thereof.

4. The endoscope apparatus according to claim 1, further comprising a first wavelength filter that passes light in the wavelength region of the first returned light and a second wavelength filter that passes light in the wavelength region of the second returned light, wherein
the light receiving section includes a first light receiving element that receives light passed by the first wavelength filter and a second light receiving element that receives light passed by the second wavelength filter.

5. The endoscope apparatus according to claim 4, wherein
a plurality of the first wavelength filters and a plurality of the second wavelength filters are arranged two-dimensionally, and
a plurality of the first light receiving elements and a plurality of the second light receiving elements are respectively arranged at positions corresponding to the first wavelength filters and the second wavelength filters.

6. The endoscope apparatus according to claim 1, further comprising an image generating section that generates images at the focal positions of the irradiation light in the target, based on the first returned light and the second returned light received by the light receiving section.

7. The endoscope apparatus according to claim 1, further comprising a light source that generates the irradiation light.

8. The endoscope apparatus according to claim 7, wherein
the light source emits the irradiation light to include light in different wavelength regions for respectively exciting different luminescent substances to emit luminescent light in different wavelength regions.

9. The endoscope apparatus according to claim 2, further comprising an injecting section that injects the luminescent substance into the target.

10. The endoscope apparatus according to claim 1, wherein
the second optical system is arranged such that the optical axis thereof has a different orientation than the optical axis of the first optical system.
